(19) United States
(12) Patent Application Publication
(10) Pub. No.: US 2025/0303170 A1
(43) Pub. Date: Oct. 2, 2025

(54) IMPLANTABLE MEDICAL SYSTEMS WITH MEDICAL LEADS WITH MULTIPLE NEURAL INTERFACES

(71) Applicant: The Alfred E. Mann Foundation for Scientic Research, Valencia, CA (US)

(72) Inventors: Brian Dearden, Pasadena, CA (US); Joseph L. Calderon, Santa Clarita, CA (US); Brian Michael Shelton, Ventura, CA (US); Neil Talbot, La Crescenta, CA (US); Anthony Ng, Santa Clarita, CA (US)

(21) Appl. No.: 19/093,145

(22) Filed: Mar. 27, 2025

Related U.S. Application Data
(60) Provisional application No. 63/572,121, filed on Mar. 29, 2024.

(51) Int. Cl. A61N 1/36; A61N 1/05; A61N 1/378

(52) U.S. Cl. CPC A61N 1/36128; A61N 1/0556; A61N 1/378

(57) ABSTRACT

An implantable medical system includes a medical device connected to a medical lead with several neural interfaces. The system can selectively generate stimulation signals at a selected one of the several neural interfaces, such as nerve cuffs. Each neural interface has at least one working electrode that is common to all the neural interfaces. Each neural interface has at least one reference electrode that is dedicated to that interface. A neural interface that has not been selected to stimulate tissue has its dedicated reference electrode floating with respect to power and ground.

### CROSS-REFERENCE TO RELATED APPLICATION

This application claims priority to, and the benefit of, U.S. Provisional Application No. 63/572,121, filed on Mar. 29, 2024, the entire content of which is hereby incorporated by reference.

### BACKGROUND

### 1. Field

The present disclosure relates to stimulation devices and methods of providing stimulation.

### 2. Description of the Related Art

Electrical stimulation can be provided to one or more nerves to treat one or more medical conditions, and it can be desirable to stimulate multiple nerves to effectively treat certain medical conditions. For example, stimulating both (e.g., simultaneously) the right and left hypoglossal nerves (HGN) may be more effective at treating obstructive sleep apnea (OSA) in patients experiencing complete concentric collapse. It can also be desirable to selectively provide stimulation to certain parts of a nerve, which can be provided by an electrical lead having a plurality of independently drivable electrodes. For example, the proximal portion of an HGN nerve includes both (1) nerve fascicles that control the protrusion of the tongue and (2) nerve fascicles that control the retraction of tongue, and it can be desirable at least for purposes of treating OSA to selectively stimulate the nerve fascicles that cause tongue protrusion without stimulating those that cause retraction.

However, a stimulation device configured to selectively stimulate each of multiple different nerves could require a substantial number of electrodes. Configuring the stimulation device to independently control each of such electrodes could increase the number of components, cost, complexity, and likelihood of component failure of a driver and/or other components of the stimulation device. Or, when the driver and/or other components are limited in the number of electrodes that can be independently controlled or selected, the stimulation device may be unable to stimulate the multiple different nerves with sufficient selectivity to effectively treat a specific medical condition. It is in view of this technical background that the present disclosure is provided.

This Background section is provided only for purpose of introducing certain background information relating to the present disclosure and, thus, statements made in this Background section are not admissions of prior art.

### SUMMARY

According to an aspect, the technology relates to an implantable medical lead including a lead body extending between a proximal end and first and second distal ends; and a plurality of conductors disposed within the lead body, wherein a first conductor of the plurality of conductors extends from the proximal end of the lead body to a first electrode at the first distal end, a second conductor of the plurality of conductors extends from the proximal end of the lead body to a first electrode at the second distal end, and a third conductor of the plurality of conductors extends from the proximal end of the lead body to a second electrode at the first distal end; and to a second electrode at the second distal end.

In some examples, the first electrode at the first distal end includes a first return electrode, the first electrode at the second distal end includes a second return electrode, the second electrode at the first distal end includes a first working electrode; and the second electrode at the second distal end includes a second working electrode.

In some examples, the first return electrode and the first working electrode at the first distal end include a first neural interface configured for neural stimulation; and the second return electrode and the second working electrode at the second distal end include a second neural interface configured for neural stimulation.

In some examples, a medical system includes the implantable medical lead; and an implantable medical device configured for generating stimulation signals, the implantable medical device including a stimulation system, including a plurality of subsystems, including a current source, a current sink, a voltage source, and a ground; a matrix switch configured to selectively couple any one of the first and second return electrodes to any one of the plurality of subsystems, and selectively couple all of the first and second working electrodes to any other one of the plurality of subsystems; and a controller configured to control the operation of the matrix switch.

In some examples, the implantable medical device is configured to generate neurostimulation signals at the first neural interface.

In some examples, the controller is configured to not switchably couple the stimulation system to the second return electrode in the second neural interface when the implantable medical device generates the neurostimulation signals at the first neural interface.

In some examples, the second return electrode in the second neural interface is floating and is not coupled to ground or any power source when the implantable medical device generates the neurostimulation signals at the first neural interface.

In some examples, the controller is configured to switchably couple the current source to the first and second working electrodes in the first and second neural interfaces via the third conductor of the plurality of conductors; and switchably couple the ground to the first return electrode in the first neural interface via the first conductor of the plurality of conductors.

In some examples, the controller is configured to switchably couple the voltage source to the first and second working electrodes in the first and second neural interfaces via the third conductor of the plurality of conductors; and switchably couple the current sink to the first return electrode in the first neural interface via the first conductor of the plurality of conductors.

In some examples, the implantable medical device is configured to generate neurostimulation signals at the second neural interface.

In some examples, the controller is configured to not switchably couple the stimulation system to the first return electrode in the first neural interface when the implantable medical device generates the neurostimulation signals at the second neural interface.

In some examples, the first return electrode is floating and is not coupled to ground or any power source when the implantable medical device generates the neurostimulation signals at the second neural interface.

In some examples, the controller is configured to switchably couple the current source to the first and second working electrodes via the third conductor of the plurality of conductors; and switchably couple the ground to the second return electrode via the second conductor of the plurality of conductors.

In some examples, the controller is configured to switchably couple the voltage source to the first and second working electrodes via the third conductor of the plurality of conductors; and switchably couple the current sink to the second return electrode via the second conductor of the plurality of conductors.

In some examples, the first neural interface includes at least one of a nerve cuff, a helical cuff, paddle electrodes or an electrode array; and the second neural interface includes at least one of a nerve cuff, a helical cuff, paddle electrodes or an electrode array.

According to an aspect, the technology relates to an implantable medical system including an implantable medical lead including a lead body; a plurality of conductors disposed within the lead body, wherein a first conductor of the plurality of conductors extends from a proximal end of the lead body to a first return electrode at a first neural interface at a first distal end of the lead body, a second conductor of the plurality of conductors extends from the proximal end of the lead body to a second return electrode at a second neural interface at a second distal end of the lead body, and a third conductor of the plurality of conductors extends from the proximal end of the lead body to a first working electrode in the first neural interface at the first distal end; and to a second working electrode in the second neural interface at the second distal end.

In some examples, the implantable medical system includes an implantable medical device configured to generate stimulation signals at either the first or the second neural interface, the implantable medical device including a stimulation system including a plurality of subsystems including a current source, a current sink, a voltage source, and a ground; a matrix switch configured to selectively couple any one of the first and second return electrodes to any one of the plurality of subsystems, and selectively couple all the first and second working electrodes to any other one of the plurality of subsystems; and a controller configured to control the operation of the matrix switch.

In some examples, the controller is configured to not switchably couple the stimulation system to the second return electrode in the second neural interface such that the second return electrode in the second neural interface is floating and is not coupled to ground or any power source, and the controller is configured to either switchably couple the current source to the first and second working electrodes in the first and second neural interfaces via the third conductor of the plurality of conductors, and switchably couple the ground to the first return electrode in the first neural interface via the first conductor of the plurality of conductors; or switchably couple the voltage source to the first and second working electrodes in the first and second neural interfaces via the third conductor of the plurality of conductors, and switchably couple the current sink to the first return electrode in the first neural interface via the first conductor of the plurality of conductors.

In some examples, the controller is configured to not switchably couple the stimulation system to the first return electrode in the first neural interface such that the first return electrode in the first neural interface is floating and is not coupled to ground or any power source, and the controller is configured to either switchably couple the current source to the first and second working electrodes in the first and second neural interfaces via the third conductor of the plurality of conductors, and switchably couple the ground to the second return electrode in the second neural interface via the second conductor of the plurality of conductors; or switchably couple the voltage source to the first and second working electrodes in the first and second neural interfaces via the third conductor of the plurality of conductors, and switchably couple the current sink to the second return electrode in the second neural interface via the second conductor of the plurality of conductors.

In some examples, the first neural interface includes at least one of a nerve cuff, a helical cuff, paddle electrodes or an electrode array; and the second neural interface includes at least one of a nerve cuff, a helical cuff, paddle electrodes or an electrode array.

According to another aspect, the technology relates to a method for configuring an implantable medical lead, including providing a lead body extending between a proximal end and first and second neural interfaces at respective first and second distal ends; providing a plurality of conductors disposed within the lead body; extending a first conductor of the plurality of conductors from the proximal end of the lead body to a first reference electrode at the first neural interface; extending a second conductor of the plurality of conductors from the proximal end of the lead body to a second reference electrode at the second neural interface; and extending a third conductor of the plurality of conductors from the proximal end of the lead body to a first working electrode at the first neural interface; and to a second working electrode at the second neural interface.

In some examples, method for configuring an implantable medical system includes the method of configuring the implantable medical lead; providing an implantable medical device, the implantable medical device including a stimulation system for generating stimulation signals via the implantable medical lead, the stimulation system including a plurality of subsystems including a current source, a current sink, a voltage source, and a ground; a matrix switch configured to selectively couple any one of the first and second reference electrodes to any one of the plurality of subsystems, and selectively couple all the first and second working electrodes to any other one of the plurality of subsystems; and a controller configured to control the operation of the matrix switch; and coupling the implantable medical lead to the implantable medical device.

In some examples, the method further includes selecting the first neural interface to receive stimulation signals from the stimulation system; coupling the first reference electrode in the first neural interface via the first conductor of the plurality of conductors to the ground via the matrix switch; selecting the second conductor of the plurality of conductors to be floating and not coupled to ground or any power source; and coupling the first and second working electrodes in the first and second neural interfaces via the third conductor of the plurality of conductors to the current source via the matrix switch.

In some examples, the method further includes selecting the first neural interface to receive stimulation signals from the stimulation system; coupling the first reference electrode in the first neural interface via the first conductor of the plurality of conductors to the current sink via the matrix switch; coupling the first and second working electrodes in the first and second neural interfaces via the third conductor of the plurality of conductors to the voltage source via the matrix switch; and selecting the second reference electrode of the second neural interface to be floating and not coupled to ground or any power source.

In some examples, the method further includes selecting the second neural interface to receive stimulation signals from the stimulation system; coupling the second reference electrode in the second neural interface via the second conductor of the plurality of conductors to the ground via the matrix switch; coupling the first and second working electrodes in the first and second neural interfaces via the third conductor of the plurality of conductors to the current source via the matrix switch; and selecting the first reference electrode of the first neural interface to be floating and not coupled to ground or any power source.

In some examples, the method further includes selecting the second neural interface to receive stimulation signals from the stimulation system; coupling the second reference electrode in the second neural interface via the second conductor of the plurality of conductors to the current sink via the matrix switch; coupling the first and second working electrodes in the first and second neural interfaces via the third conductor of the plurality of conductors to the voltage source via the matrix switch; and selecting the first reference electrode of the first neural interface to be floating and not coupled to ground or any power source.

This Summary section introduces some features of nonlimiting and non-exhaustive examples of the present disclosure, and is not intended to limit the scope of the claims.

### BRIEF DESCRIPTION OF THE DRAWINGS

The drawings, together with the specification, illustrate nonlimiting and non-exhaustive example embodiments of the present disclosure.

FIG. 1 is a block diagram of a medical system according to one or more embodiments.

FIG. 2 is a block diagram of a stimulation system according to one or more embodiments.

FIG. 3 is a block diagram of a system including a medical lead with selective connections to various subsystems according to one or more embodiments.

FIG. 4 depicts a circuit diagram of electrical connections of electrodes of the first and second cuff electrodes of the stimulation device of FIG. 1 according to one or more embodiments.

FIG. 5 is a flow chart for a method for coupling of a medical lead to a selected neural interface according to one or more embodiments.

FIG. 6 depicts the stimulation system of FIG. 1 when implanted in a body.

FIG. 7 depicts another stimulation system according to one or more embodiments and when implanted in a body.

### DETAILED DESCRIPTION

Nonlimiting and non-exhaustive embodiments of stimulation devices and of methods of stimulation will now be described in more detail with reference to the drawings.

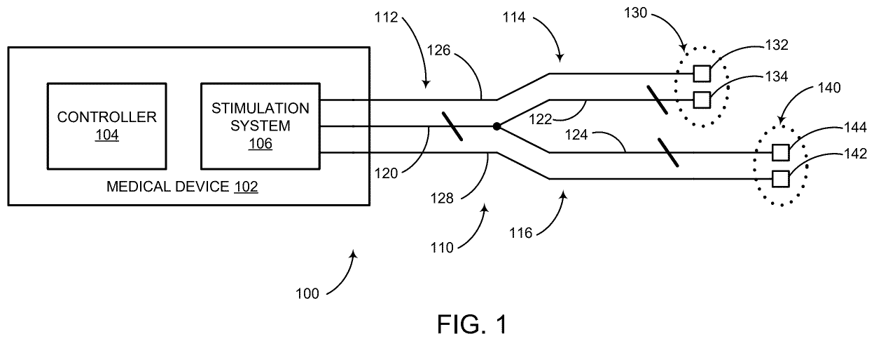

FIG. 1 is a block diagram of medical system 100 for generating neural stimulation signals applied to one or two nerves via neural interfaces 130 or 140, according to one or more embodiments. Neural interfaces 130 and 140 may be nerve cuffs, paddle electrodes, electrode arrays or other interfaces used for stimulating nerves. Medical system 100 includes medical device 102 connected to implantable medical lead 110. Medical device 102 may be an implantable pulse generator (IPG) and includes controller 104 and stimulation system 106. Controller 104 may include nonvolatile memory storing tissue stimulation protocols determined by a clinician using a clinician programmer for a patient. The tissue stimulation protocols may also be selected by a patient using a patient remote from a preselected set determined by a clinician. Controller 104 generates control signals for stimulation system 106 based on a selected stimulation protocol and selects either neural interface 130 or 140 to receive stimulation signals at any given time. Not shown in FIG. 1 are other systems typically part of implantable medical devices, such as systems to receive wireless power transmissions from external power transmitters, power supplies and systems to communicate with patient remotes and clinician programmers. However, it will be understood that some embodiments of medical systems described herein may include such other systems.

Medical lead 110 has a main section 112, which then splits into two branches 114 and 116. Branch 114 connects to neural interface 130 at one distal end and branch 116 connects to neural interface 140 at another distal end. Main section 112 has N+2 conductors at its proximal end and is connected to stimulation system 106 in medical device 102, where N can be any integer greater than or equal to one. Branch 114 has conductor 126 connected to reference electrode 132 in neural interface 130 and also N conductors 120 connected to N working electrodes 134 in neural interface 130. Branch 116 has conductor 128 connected to reference electrode 142 in neural interface 140 and also N conductors 120 connected to N working electrodes 144 in neural interface 140.

Stimulation system 100 may be a subcutaneously implantable stimulation device and include an implantable pulse generator (IPG) and one or more leads (e.g., electrical leads) electrically coupled to the IPG and configured to provide stimulation (e.g., electrical stimulation). The stimulation device 100 may be implanted in a body (e.g., a human or a non-human animal) and utilized to stimulate, for example, one or more nerves to treat one or more conditions. For example, the stimulation system 100 may be utilized to stimulate two hypoglossal nerves on opposite sides of a sagittal plane of the body in order to treat obstructive sleep apnea (OSA), including OSA where complete concentric collapse (CCC) occurs. In another example, the stimulation system 100 may be used to selectively stimulate another nerve (e.g., a vagus nerve and/or other nerves) in addition to, or instead of, the hypoglossal nerve to treat one or more medical conditions.

The one or more leads may include, for example, a bifurcated lead including a common lead 110 electrically coupled to the medical device 102 at a proximal end, a first branch 114 branching off from a distal end of the common lead 110, and a second branch 116 branching off from the distal end of the common lead 110. The first branch 114 may include a first neural interface 130, such as a cuff electrode, and the second branch 116 may include a second neural interface 140, such as a cuff electrode.

However, the present disclosure is not limited thereto. For example, the lead may be a multi-furcated lead that includes two or more branches or sub-leads that, for example, branch off from a common lead and each include one or more electrodes. The one or more electrodes on each sub-lead may be configured (shaped, sized, relatively positioned, relatively oriented, and/or of a number) to stimulate one or more nerves (e.g., the proximal and/or distal portion of the hypoglossal nerve). Because the shape, size, and position of nerves vary, the shape, size, relative positions, relative orientations, and/or number of the one or more electrodes along each branch or sub-lead may vary based on the particular nerve that the one or more electrodes are configured to stimulate. Moreover, in one or more embodiments, each branch or sub-lead may include one or more stimulators other than electrodes, such as one or more coils for generating a time-varying magnetic field, one or more acoustic stimulators, etc.

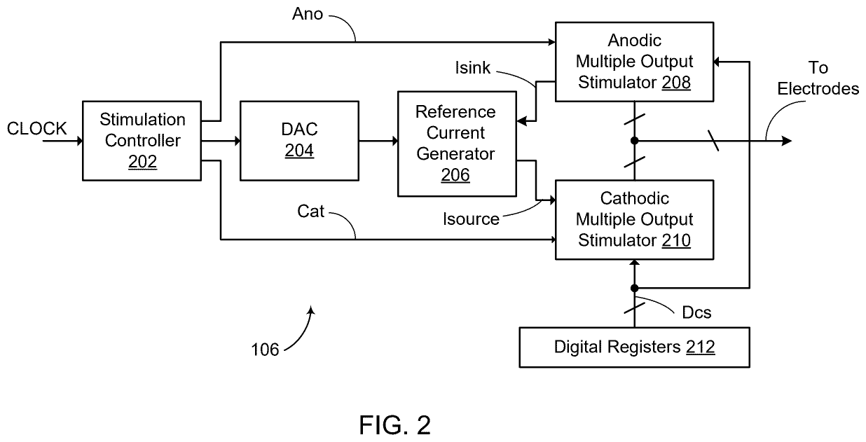

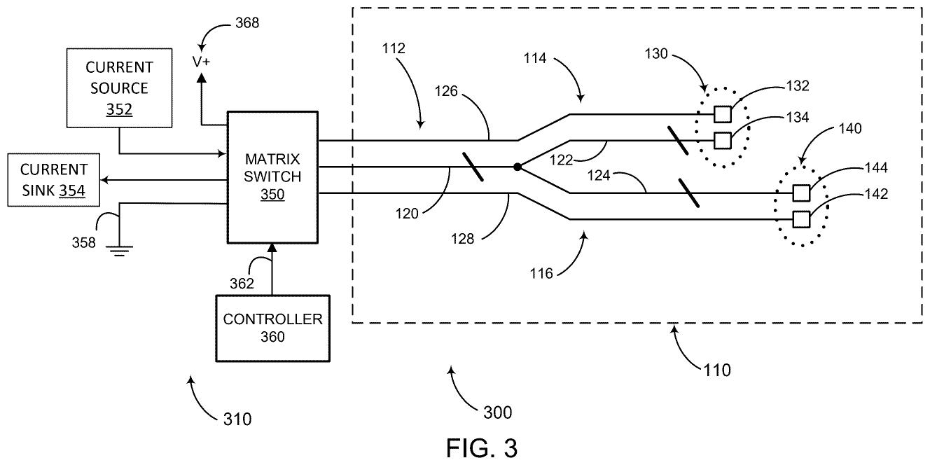

FIG. 2 is a block diagram of an exemplary stimulation system 106 connected by switches to the conductors of medical lead 110 according to one or more embodiments. Stimulation system 106 is similar to the pulse generator shown in FIG. 3 in U.S. Pat. No. 9,446,241. Stimulation controller 202 receives control signals from controller 104 via signal lines, which are not shown in FIG. 2. Digital control signals from stimulation controller 202 are sent to anodic stimulator 208, cathodic stimulator 210 and digital to analog converter DAC 204. Stimulation system 106 provides for selecting either neural interface 130 or 140 and for selecting an anodic or cathodic stimulator based upon tissue stimulation requirements determined by a clinician.

The outputs of the anodic stimulators 208 and cathodic stimulators 210 are selected by stimulation controller 202 by setting the corresponding “bits” in digital registers 212. Control lines from stimulation controller 202 to digital registers 212 are not shown in FIG. 2. Digital registers 212 generate digital control signals Des, which control the selection of either neural interface 130 or 140 to provide stimulation signals to tissue by selecting via switches either of the respective reference electrodes 132 or 142 via the selection of either respective conductor 126 or 128 of medical lead 110.

Digital registers 212 also store information regarding stimulation pulse duration, amplitude and profile as well as other operational parameters. Based upon information stored in digital registers 212 and the Clock signal, stimulation controller 202 generates the desired stimulation pulse amplitude and triggers digital to analog converter DAC 204 to generate an output. Based upon the DAC 204 output, reference current source generator 206 provides a current sink for Isink current for the anodic stimulator 208 and provides a current source Isource current for the cathodic stimulator 210. Stimulation controller 202 generates control signal Ano to turn on the anodic stimulator 208 to output anodic current at one or more selected outputs according to the programmed anodic pulse amplitude, duration and profile. Anodic stimulator 208 may include one or more normally open switches. Similarly, stimulation controller 202 also generates control signal Cat to turn on cathodic stimulator 210 to output cathodic current at one or more selected outputs according to the programmed cathodic pulse amplitude, duration and profile. Stimulation system 106 is connected to medical lead 110.

FIG. 3 is a block diagram of a system 300 including medical lead 110 with switchable selective connections via matrix switch 350 to various subsystems 310 including current source 352, current sink 354, voltage source 368 and ground 358 according to one or more embodiments. System 300 includes medical lead 110 connected to subsystems 310, which is a portion of a stimulation system with control signals coming from controller 360. Controller 360 generates control signals for the stimulation system based on a selected stimulation protocol and selects either neural interface 130 or 140 to receive stimulation signals.

In one or more embodiments, stimulation controller 360 may select neural interface 130 or 140 for tissue stimulation and can generate control signals on control line 362 to matrix switch 350 to connect current source 352 to the N conductors 120 to the working electrodes 134 and 144. Controller 360 can also generate control signals on control line 362 to matrix switch 350 to connect ground 358 to reference electrode 132 via conductor 126 to enable neural interface 130 for stimulation of neural tissue.

In other embodiments, controller 360 can generate control signals on control line 362 to matrix switch 350 to connect ground 358 to reference electrode 142 via conductor 128 to enable neural interface 140 for stimulation of neural tissue Conductor 120 may have N conductors, where N is an integer greater than or equal to one. As a result of switching functions provided by matrix switch 350 either neural interface 130 or 140 may be activated to stimulate tissue.

When neural interface 130 is delivering stimulation signals to tissue, neural interface 140 is not functional, because reference electrode 142 is floating, since it is not connected to ground or a power source. Neural interfaces 130 and 140 are at separate distal ends of medical lead 110 and separated by a distance sufficient to prevent reference electrode 142 from acting as a reference electrode to working electrodes 134 because of a relatively high impedance path between reference electrode 142 and working electrodes 134.

In one or more embodiments, stimulation controller may select neural interface 130 or 140 for tissue stimulation and can generate control signals via control line 362 to matrix switch 350 to connect voltage source 368 to working electrodes 134 and 144 via N conductors 120. Controller 360 can also generate control signals via control line 362 to matrix switch 350 to connect current sink 354 to reference electrode 142 via conductor 128 to activate neural interface 140 for stimulation of neural tissue.

In other embodiments, controller 360 can generate control signals via control line 362 to matrix switch 350 to connect current sink 354 to reference electrode 132 via conductor 126 to activate neural interface 130 for stimulation of neural tissue. Conductor 120 may have N conductors, where N is an integer greater than or equal to one.

When neural interface 140 is delivering stimulation signals to tissue, neural interface 130 is not functional, because reference electrode 132 is floating, since it is not connected to ground or a power source. Neural interfaces 130 and 140 are at separate distal ends of medical lead 110 and separated by a distance sufficient to prevent reference electrode 132 from acting as a reference electrode to working electrodes 144 because of a relatively high impedance path between reference electrode 132 and working electrodes 144.

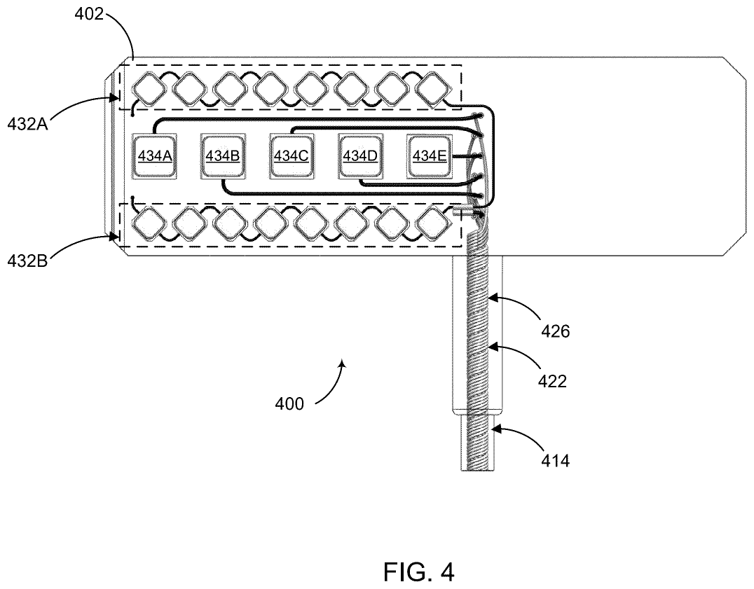

FIG. 4 depicts a nerve cuff 400 connected to branch 414 of a medical lead according to one or more embodiments. Branch 414 includes N conductors 422 to working electrodes 434A-434E and conductor 426 to arrays of reference electrodes 432A and 432B. The electrodes are disposed on flexible base 402. N can be an integer equal to or greater than one. In some embodiments, working electrodes 434A-434E can be connected together as one electrode. In some embodiments, working electrodes 434A-434E can each be connected to separate conductors within the N conductors 422 of branch 414, and provide separate N channels of stimulation signals.

Medical system 100, in one or more embodiments, includes medical device 102 and medical lead 110 and can be configured to be connected to one nerve cuff, like nerve cuff 400, at a first distal end and functioning as neural interface 130 and connected to a second nerve cuff, like nerve cuff 400, at a second distal end and functioning as neural interface 140.

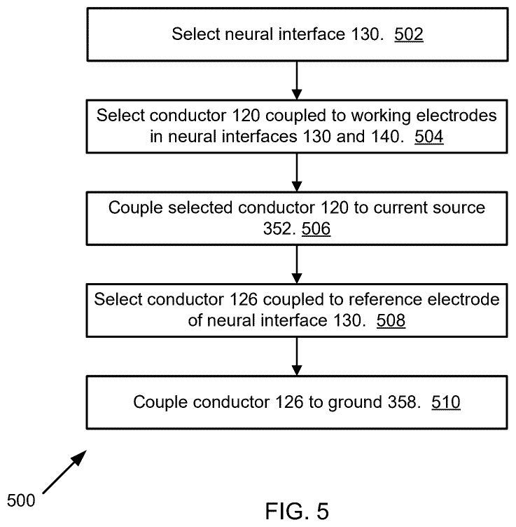

FIG. 5 is a flow chart for method 500 for selective coupling of medical lead 110 to neural interface 130 for system 300 in FIG. 3 according to one or more embodiments. Step 502 of the method selects neural interface 130 to receive stimulation signals. Step 504 of the method selects working electrodes 134 and 144 via conductor 120. Step 506 connects conductor 120 to current source 352. Step 508 selects conductor 126 coupled to reference electrode 132 of neural interface 130. Step 510 connects conductor 126 and reference electrode 132 to ground 358. Neural interface 130 is coupled to current source 352 and ground 358 and is configured to receive stimulation signals from medical device 102.

At step 510, neural interface 140 is not functional because reference electrode 142 of neural interface 140 is floating since it is not coupled to ground or a power source. Reference electrode 142 cannot function as a reference electrode to working electrodes 134 because of a relatively high impedance path between reference electrode 142 and working electrodes 134.

Method 500 provides steps for the stimulation during a first time period of tissue using neural interface 130, while neural interface 140 is not functional.

When both work and return electrodes on a same lead are driven together, stimulation provided by the lead to a nerve in proximity to the lead may be, for example, bipolar or tripolar stimulation, because both anode and cathode electrodes on the same lead are driven concurrently (e.g., simultaneously). In contrast, when only one of work or return electrodes on a same lead are driven (without the other), then the stimulation provided by the lead may be monopolar stimulation, because only one of cathode electrodes or anode electrodes are driven (without the other). Monopolar stimulation is generally much more diffuse and can be insufficient in intensity to stimulate the nearby nerve.

The method 500 for providing stimulation using the neural interface 130 without providing stimulation using the neural interface 140 may define a first stimulation mode of the stimulation system 106. A corresponding method may be used by the stimulation system 106 to provide stimulation using the neural interface 140 without providing stimulation using the neural interface 130, and this corresponding method may define a second stimulation mode of the stimulation system 106. In some embodiments, the stimulation system 106 is configured to utilize the second stimulation mode during a second time period after (e.g., immediately after) the first time period.

The controller 104 may be configured, when executing instructions stored in the memory, not shown in the figures, to control the stimulation system 106 to alternatingly (e.g., alternatingly at a set frequency, such as a frequency equal to or greater than 1 Hz, 10 Hz, 60 Hz, or 100 Hz) utilize the first and second stimulation modes to drive the first and second branches 114 and 116. For example, the controller 104 may be configured to control the stimulation system 106 during a third time period after (e.g., immediately after) the second time period to drive the first and second branches 114 and 116 according to the first stimulation mode.

The present disclosure is not limited by the stimulation device 100 and stimulation methods of FIGS. 1-5. Although the stimulation device 100 is illustrated to include two branches, the present disclosure is not limited thereto. The stimulation device 100 may include, for example, three or more branches (e.g., each branching off from the common lead 110), and each branch may include, for example, one or more working electrodes (e.g., 1, 2, 3, 4, 5, or more working electrodes) and one or more return electrodes (e.g., 1, 2, 3, 4, 5, or more return electrodes).

In one or more embodiments, the controller 104 may be configured, when executing instructions stored in memory, to control the stimulation system 106 to sequentially drive return electrodes on the sub leads so that only one of the three or more branches has its respective return electrode(s) driven at a time, while the return electrode(s) of the remaining sub leads are not driven, in a similar manner as is described herein above. For example, the stimulation system 100 may include three branches, and the stimulation system 106 may drive three branches in a first stimulation mode during a first time period, drive the three branches in a second stimulation mode during a second time period after the first time period, drive the three branches in a third stimulation mode during a third time period after the second time period, and drive the three branches in the first stimulation mode during a fourth time period after the third time period.

In one or more embodiments, a single bifurcated lead (or a single branch) may include the first and second neural interfaces 130 and 140 respectively at two positions along the lead that are separated from each other.

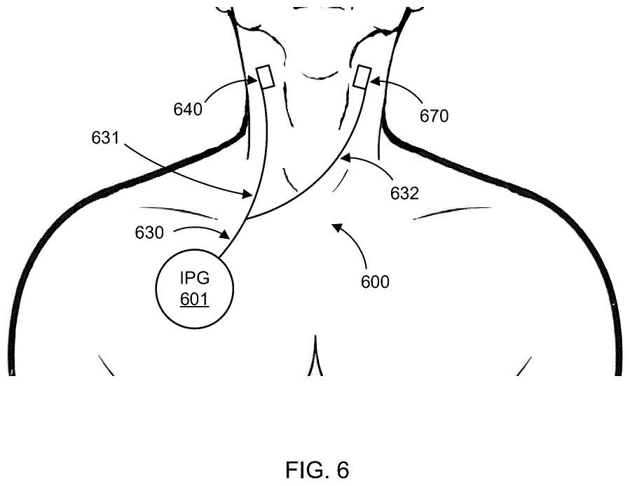

Referring to FIG. 6, the stimulation device 600 may be an implantable stimulation device, for example, for providing stimulation to multiple nerves and/or muscles to treat one or more medical conditions. The stimulation device 600 (e.g., the housing 601 and at least the exterior surfaces of the common lead 630, the first branch or sub lead 631, and the second branch or sub lead 632) may include (e.g., may be made of) a biocompatible material. The work and return electrodes on each sub lead may each be positioned within the body proximal to (e.g., be around or at least partially surrounding) a target nerve and/or muscle, such as a proximal or distal portion of the HGN or other nerve. For example, the first and second cuff electrodes 640 and 670 may be respectively positioned to stimulate first and second HGN nerves on opposite sides of a sagittal plane of the body.

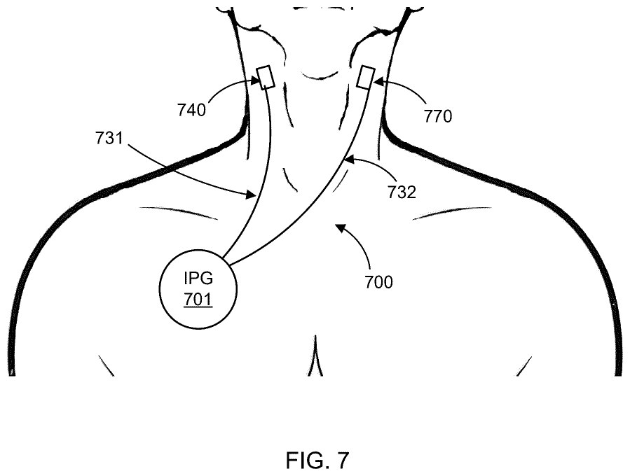

FIG. 7 depicts another stimulation device 700 according to one or more embodiments and when implanted in a body. The stimulation device 700 may include features similar to, or the same as, the stimulation device 600 shown in FIG. 6. For example, the stimulation device 700 may include an IPG including a housing 701 and a power source, a power modulation electronics, a driver, a memory, and a controller within the housing 701. The stimulation device 700 may include a first lead 731 including a first cuff electrode 740, and a second lead 732 including a second cuff electrode 770. The stimulation device 700 may differ from the first stimulation device 600 in that the first and second leads 731 and 732 are separately coupled to the IPG, instead of branching off from a common lead that is coupled to the IPG.

In one or more embodiments a stimulation device and/or a method of stimulation may be utilized to treat one or more medical conditions by stimulating two or more nerves. For example, the OSA (including OSA where complete concentric collapse occurs), dysphagia, etc. may be treated. OSA where complete concentric collapse occurs may be treated, for example, by stimulating (e.g., alternatingly stimulating) two HGN nerves on opposite sides of a sagittal plane. As another example, the stimulation device and/or a method of stimulation may be utilized to treat epilepsy or depression, for example, by stimulating the vagus nerve at two or more discrete locations or in conjunction with another nerve.

Although some methods for providing stimulation have been discussed with reference to FIG. 5, the present disclosure is not limited thereto. Stimulation devices for providing stimulation, and processes performed by such stimulation devices, have been described herein with reference to FIGS. 1-7, and the present disclosure includes all methods for providing stimulation that include any combination of such processes in any suitable order.

It will be understood that, although the terms “first”, “second”, “third”, etc., may be utilized herein to describe one or more suitable features, elements, or processes, these features, elements, or processes should not be limited by these terms. These terms are only utilized to distinguish one feature, element, or process from another feature, element, or process. Thus, a first feature, element, or process discussed herein could be termed a second feature, element, or process without departing from the spirit and scope of the present disclosure.

The terminology utilized herein is for the purpose of describing particular embodiments only and is not intended to be limiting of the present disclosure. As utilized herein, the singular forms “a”, “an” and “the” are intended to include the plural forms as well, unless the context clearly indicates otherwise. It will be further understood that the terms “comprises,” “comprising,” “includes,” and “including,” when utilized in this specification, specify the presence of stated features, elements, and/or processes, but do not preclude the presence or addition of one or more other features, elements, and/or processes. As utilized herein, the term “and/or” includes any and all combinations of one or more of the associated listed items. Expressions such as “at least one of,” when preceding a list of elements, modify the entire list of elements and do not modify the individual elements of the list. Further, the use of “may” when describing embodiments of the present disclosure refers to “one or more embodiments of the present disclosure.”

It will be understood that when an element or feature is referred to as being “on” or “coupled to” another element or feature, it can be directly on or coupled to the other element or feature, or one or more intervening element(s) or feature(s) may be present. In contrast, when an element or feature is referred to as being “directly on,” or “directly coupled to” another element or feature, there are no intervening elements or features present.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The stimulation devices (e.g., the stimulation system 100) and/or any relevant components of the stimulation devices (e.g., the controller 104, the memory, the stimulation system 106, etc.) within the scope of the present disclosure may be implemented utilizing any suitable circuits, hardware (e.g. discrete electronic components or an application-specific integrated circuit), firmware, software, or a combination of software, firmware, hardware, and circuits. For example, the one or more suitable components of the stimulation devices may be formed on one integrated circuit (IC) chip or on separate IC chips. Further, the one or more suitable components of the stimulation devices may be implemented on a flexible printed circuit film, a tape carrier package (TCP), a printed circuit board (PCB), or formed on one substrate. Further, the one or more suitable components of the stimulation devices may be a process or thread, running on one or more processors, in one or more computing devices, executing computer program instructions and interacting with other system components for performing the one or more suitable functionalities described herein. The computer program instructions may be stored in a memory which may be implemented in a computing device utilizing a standard memory device, such as, for example, a random access memory (RAM). The computer program instructions may also be stored in other non-transitory computer readable media such as, for example, a CD-ROM, flash drive, and/or the like. Also, a person of skill in the art should recognize that the functionality of one or more suitable computing devices may be combined or integrated into a single computing device, or the functionality of a particular computing device may be distributed across one or more other computing devices without departing from the scope of the example embodiments of the present disclosure.

Although specific embodiments are described herein, the scope of the technology is not limited to those specific embodiments. Moreover, while different examples and embodiments may be described separately, such embodiments and examples may be combined with one another in implementing the technology described herein. One skilled in the art will recognize other embodiments or improvements that are within the scope and spirit of the present technology. Therefore, the specific embodiments illustrated and described herein are only examples, and the scope of the present disclosure encompasses additional embodiments. The scope of the technology is defined by the following claims and any equivalents thereof.

What is claimed is:

1. An implantable medical lead comprising:
a lead body extending between a proximal end and first and second distal ends; and
a plurality of conductors disposed within the lead body, wherein:
a first conductor of the plurality of conductors extends from the proximal end of the lead body to a first electrode at the first distal end,
a second conductor of the plurality of conductors extends from the proximal end of the lead body to a first electrode at the second distal end, and
a third conductor of the plurality of conductors extends from the proximal end of the lead body:
to a second electrode at the first distal end; and
to a second electrode at the second distal end.

2. The implantable medical lead of claim 1, wherein:
the first electrode at the first distal end comprises a first return electrode,
the first electrode at the second distal end comprises a second return electrode,
the second electrode at the first distal end comprises a first working electrode; and
the second electrode at the second distal end comprises a second working electrode.

3. The implantable medical lead of claim 2, wherein:
the first return electrode and the first working electrode at the first distal end comprise a first neural interface configured for neural stimulation; and
the second return electrode and the second working electrode at the second distal end comprise a second neural interface configured for neural stimulation.

4. A medical system, comprising:
the implantable medical lead of claim 3; and
an implantable medical device configured for generating stimulation signals, the implantable medical device comprising a stimulation system, comprising:
a plurality of subsystems, comprising:
a current source,
a current sink,
a voltage source, and
a ground;
a matrix switch configured to:
selectively couple any one of the first and second return electrodes to any one of the plurality of subsystems, and
selectively couple all of the first and second working electrodes to any other one of the plurality of subsystems; and
a controller configured to control the operation of the matrix switch.

5. The medical system of claim 4, wherein the implantable medical device is configured to generate neurostimulation signals at the first neural interface.

6. The medical system of claim 5, wherein the controller is configured to not switchably couple the stimulation system to the second return electrode in the second neural interface when the implantable medical device generates the neurostimulation signals at the first neural interface.

7. The medical system of claim 5, wherein the second return electrode in the second neural interface is floating and is not coupled to ground or any power source when the implantable medical device generates the neurostimulation signals at the first neural interface.

8. The medical system of claim 5, wherein the controller is configured to:
switchably couple the current source to the first and second working electrodes in the first and second neural interfaces via the third conductor of the plurality of conductors; and
switchably couple the ground to the first return electrode in the first neural interface via the first conductor of the plurality of conductors.

9. The medical system of claim 5, wherein the controller is configured to:
switchably couple the voltage source to the first and second working electrodes in the first and second neural interfaces via the third conductor of the plurality of conductors; and
switchably couple the current sink to the first return electrode in the first neural interface via the first conductor of the plurality of conductors.

10. The medical system of claim 4, wherein the implantable medical device is configured to generate neurostimulation signals at the second neural interface.

11. The medical system of claim 10, wherein the controller is configured to not switchably couple the stimulation system to the first return electrode in the first neural interface when the implantable medical device generates the neurostimulation signals at the second neural interface.

12. The medical system of claim 10, wherein the first return electrode is floating and is not coupled to ground or any power source when the implantable medical device generates the neurostimulation signals at the second neural interface.

13. The medical system of claim 10, wherein the controller is configured to:
switchably couple the current source to the first and second working electrodes via the third conductor of the plurality of conductors; and
switchably couple the ground to the second return electrode via the second conductor of the plurality of conductors.

14. The medical system of claim 10, wherein the controller is configured to:
switchably couple the voltage source to the first and second working electrodes via the third conductor of the plurality of conductors; and
switchably couple the current sink to the second return electrode via the second conductor of the plurality of conductors.

15. The implantable medical lead of claim 3, wherein the first neural interface comprises at least one of a nerve cuff, a helical cuff, paddle electrodes or an electrode array; and the second neural interface comprises at least one of a nerve cuff, a helical cuff, paddle electrodes or an electrode array.

16. An implantable medical system comprising:
an implantable medical lead comprising:
a lead body;
a plurality of conductors disposed within the lead body, wherein:
a first conductor of the plurality of conductors extends from a proximal end of the lead body to a first return electrode at a first neural interface at a first distal end of the lead body,
a second conductor of the plurality of conductors extends from the proximal end of the lead body to a second return electrode at a second neural interface at a second distal end of the lead body, and
a third conductor of the plurality of conductors extends from the proximal end of the lead body:
to a first working electrode in the first neural interface at the first distal end; and
to a second working electrode in the second neural interface at the second distal end.

17. The implantable medical system of claim 16 further comprising:
an implantable medical device configured to generate stimulation signals at either the first or the second neural interface, the implantable medical device comprising:
a stimulation system comprising:
a plurality of subsystems comprising:
a current source,
a current sink,
a voltage source, and
a ground;
a matrix switch configured to:
selectively couple any one of the first and second return electrodes to any one of the plurality of subsystems, and
selectively couple all the first and second working electrodes to any other one of the plurality of subsystems; and
a controller configured to control the operation of the matrix switch.

18. The implantable medical system of claim 17:
wherein the controller is configured to not switchably couple the stimulation system to the second return electrode in the second neural interface such that the second return electrode in the second neural interface is floating and is not coupled to ground or any power source, and
wherein the controller is configured to either:
switchably couple the current source to the first and second working electrodes in the first and second neural interfaces via the third conductor of the plurality of conductors, and
switchably couple the ground to the first return electrode in the first neural interface via the first conductor of the plurality of conductors; or
switchably couple the voltage source to the first and second working electrodes in the first and second neural interfaces via the third conductor of the plurality of conductors, and
switchably couple the current sink to the first return electrode in the first neural interface via the first conductor of the plurality of conductors.

19. The implantable medical system of claim 17:
wherein the controller is configured to not switchably couple the stimulation system to the first return electrode in the first neural interface such that the first return electrode in the first neural interface is floating and is not coupled to ground or any power source, and
wherein the controller is configured to either:
switchably couple the current source to the first and second working electrodes in the first and second neural interfaces via the third conductor of the plurality of conductors, and
switchably couple the ground to the second return electrode in the second neural interface via the second conductor of the plurality of conductors; or
switchably couple the voltage source to the first and second working electrodes in the first and second neural interfaces via the third conductor of the plurality of conductors, and
switchably couple the current sink to the second return electrode in the second neural interface via the second conductor of the plurality of conductors.

20. The implantable medical system of claim 16, wherein the first neural interface comprises at least one of a nerve cuff, a helical cuff, paddle electrodes or an electrode array; and the second neural interface comprises at least one of a nerve cuff, a helical cuff, paddle electrodes or an electrode array.

21. A method for configuring an implantable medical lead, comprising:
providing a lead body extending between a proximal end and first and second neural interfaces at respective first and second distal ends;
providing a plurality of conductors disposed within the lead body;
extending a first conductor of the plurality of conductors from the proximal end of the lead body to a first reference electrode at the first neural interface;
extending a second conductor of the plurality of conductors from the proximal end of the lead body to a second reference electrode at the second neural interface; and
extending a third conductor of the plurality of conductors from the proximal end of the lead body:
to a first working electrode at the first neural interface; and
to a second working electrode at the second neural interface.

22. A method for configuring an implantable medical system, the method comprising:
the method of claim 21;
providing an implantable medical device, the implantable medical device comprising:
a stimulation system for generating stimulation signals via the implantable medical lead, the stimulation system comprising:
a plurality of subsystems comprising:
a current source,
a current sink,
a voltage source, and
a ground;
a matrix switch configured to:
selectively couple any one of the first and second reference electrodes to any one of the plurality of subsystems, and
selectively couple all the first and second working electrodes to any other one of the plurality of subsystems; and
a controller configured to control the operation of the matrix switch; and
coupling the implantable medical lead to the implantable medical device.

23. The method of claim 22 and further comprising:
selecting the first neural interface to receive stimulation signals from the stimulation system;
coupling the first reference electrode in the first neural interface via the first conductor of the plurality of conductors to the ground via the matrix switch;
selecting the second conductor of the plurality of conductors to be floating and not coupled to ground or any power source; and
coupling the first and second working electrodes in the first and second neural interfaces via the third conductor of the plurality of conductors to the current source via the matrix switch.

24. The method of claim 22 and further comprising:
selecting the first neural interface to receive stimulation signals from the stimulation system;
coupling the first reference electrode in the first neural interface via the first conductor of the plurality of conductors to the current sink via the matrix switch;
coupling the first and second working electrodes in the first and second neural interfaces via the third conductor of the plurality of conductors to the voltage source via the matrix switch; and
selecting the second reference electrode of the second neural interface to be floating and not coupled to ground or any power source.

25. The method of claim 22 and further comprising:
selecting the second neural interface to receive stimulation signals from the stimulation system;
coupling the second reference electrode in the second neural interface via the second conductor of the plurality of conductors to the ground via the matrix switch;
coupling the first and second working electrodes in the first and second neural interfaces via the third conductor of the plurality of conductors to the current source via the matrix switch; and
selecting the first reference electrode of the first neural interface to be floating and not coupled to ground or any power source.

26. The method of claim 22 and further comprising:
selecting the second neural interface to receive stimulation signals from the stimulation system;
coupling the second reference electrode in the second neural interface via the second conductor of the plurality of conductors to the current sink via the matrix switch;
coupling the first and second working electrodes in the first and second neural interfaces via the third conductor of the plurality of conductors to the voltage source via the matrix switch; and
selecting the first reference electrode of the first neural interface to be floating and not coupled to ground or any power source.
